(19) United States
(12) Patent Application Publication
(10) Pub. No.: US 2020/0388278 A1
(43) Pub. Date: Dec. 10, 2020

(54) HEARING PROTECTION SYSTEMS

(71) Applicant: Craig D. Gates, Mead, WA (US)

(72) Inventor: Craig D. Gates, Mead, WA (US)

(21) Appl. No.: 16/432,380

(22) Filed: Jun. 5, 2019

(51) Int. Cl. G10L 15/22; A61F 11/08

(52) U.S. Cl. CPC G10L 15/22; A61F 2011/085; A61F 11/08

(57) ABSTRACT

A hearing protection system including an earplug displaceable from an open position to a closed position in response to a signal received from a trigger mechanism. The trigger mechanism responsive to a voice input, to cause the earplug to displace between the open position and the closed position based at least in part on receiving a predetermined keyword.

### BACKGROUND

Protecting a hearing of a person may be difficult in conditions where hearing protection is desired at a time of an event but not desired until the event. This is particularly true for shooters, where hearing protection is not desired until the firing of a firearm. For example, a hunter may desire hearing protection at the time of firing a firearm, but the hearing protection is not desired until the firing of the firearm so that the hunter may hear ambient sound naturally and unchanged for better hunting.

A person may use a variety of hearing protection devices to protect the persons hearing, however each of the hearing protection devices have disadvantages. For example, earplugs may be used, however the earplugs remain stuck in the ears of a user even when the earplugs are not needed and thus prevent the person from hearing ambient sound. For example, a hunter may plug his ears with earplugs to protect his ears at a time of firing a firearm, but the earplugs prevent the hunter from hearing ambient sounds like a location of game, a location of a dog, or verbal communication from another hunter. In another example, noise cancelling earplugs may be used, however the noise cancelling earplugs do not allow the user to hear unchanged ambient sound. Instead, noise cancelling earplugs inject reproduced ambient sound into the ears which is not the same as hearing the ambient sound naturally. For example, the noise cancelling earplugs receive ambient sound and inject digitally reproduced sound into the ears of the user that is distorted and thus not the same as the natural ambient sounds. Moreover, the earplugs are uncomfortable to use because the earplugs prevent heat from dissipating from the ears, causing the ears to perspire and the user to overheat.

Accordingly, there remains a need in the art for a hearing protection system, allowing the user to hear ambient sound naturally and more comfortably, until the hearing protection system is needed to protect the ears from a loud noise.

### SUMMARY

Hearing protection systems are configured to protect a hearing of a user from a loud noise. Generally, the hearing protection systems include earplugs that are displaced between an open position and a closed position based on a position of a device. When in the open position, the earplugs are positioned away from the ears of the user to allow the user to hear ambient sound naturally at an unchanged volume, and when in the closed position, the earplugs are positioned proximal to the ears of the user to at least partly block the ambient sound and allow the user to hear the ambient sound at a reduced volume that is less than the unchanged volume. This summary is provided to introduce simplified concepts of hearing protection systems, which are further described below in the Detailed Description. This summary is not intended to identify essential features of the claimed subject matter, nor is it intended for use in determining the scope of the claimed subject matter.

In an embodiment, a hearing protection system includes an earplug displaceable between an open position and a closed position, and a trigger mechanism, responsive to a voice input, to cause the earplug to displace between the open position and the closed position based at least in part on receiving a predetermined keyword.

In another embodiment, a hearing protection system includes an earplug attachable proximate to an ear of a user. The hearing protection system also includes a trigger mechanism, responsive to a voice input, to cause the earplug to displace between an open position and a closed position based at least in part on receiving a predetermined keyword.

In another example, a hearing protection system includes an earplug displaceable between an open position and a closed position in response to a signal received from a trigger mechanism. The trigger mechanism being responsive to a voice input, to cause the earplug to displace between the open position and the closed position based at least in part on receiving a predetermined keyword.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 illustrates an example hunting environment involving hunters.

FIG. 2 illustrates a perspective view of an example hearing protection system in an open position that allows the hunters to hear ambient sound at an unchanged volume.

FIG. 3 illustrates a perspective view of the example hearing protection system shown in FIG. 2 in a closed position that allows the hunters to hear ambient sound at a reduced volume that is less than the unchanged volume.

FIG. 4 illustrates a side view an example hearing protection system in an open position that allows the hunters to hear ambient sound at an unchanged volume.

FIG. 5 illustrates a side view of the example hearing protection system shown in FIG. 4 in a closed position that allows the hunters shown in FIG. 1 to hear ambient sound at a reduced volume that is less than the unchanged volume.

FIG. 6 illustrates a back view of the example hearing protection system shown in FIG. 5 in the closed position.

FIG. 7 illustrates a top view of another example hearing protection system.

FIG. 8 illustrates a schematic view of an example trigger mechanism according to an embodiment of the instant disclosure.

FIG. 9 illustrates an example method of displacing an earplug between an open position and a closed position according to an embodiment of the instant disclosure.

FIG. 10 illustrates another example method of displacing an earplug between an open position and a closed position according to an embodiment of the instant disclosure.

FIG. 11 illustrates another example method of displacing an earplug between an open position and a closed position according to an embodiment of the instant disclosure.

### DETAILED DESCRIPTION

### Overview

This disclosure is directed to hearing protection systems that displace from an open position to a closed position to protect a person's hearing. For example, the hearing protection systems may include a trigger mechanism, responsive to a voice input, to cause the earplug to displace between the open position and the closed position based at least in part on receiving a predetermined keyword. For example, the trigger mechanism may cause the hearing protection systems to displace to the closed position in response to receiving a predetermined keyword from a voice input of a hunter. Stated otherwise, the trigger mechanism may base the closing criteria on a predetermined keyword voiced by a hunter. Moreover, the trigger mechanism may employ a microphone and a microcontroller to continually respond to a voice input of a hunter. The trigger mechanism may cause the hearing protection systems to displace to the closed position where the hearing protection systems are positioned proximal to the ears of the hunter. In this way, the hearing protection systems at least partly close the ears of the hunter when the firearm is fired to prevent injury to the ears of the hunter. While this application describes various embodiments of hearing protection systems used in the field of hunting, this is by way of example and not limitation. For example, the hearing protection systems may be used in other fields such as military applications, construction applications, industrial applications, heavy equipment operation applications, machinery operation applications, music industry applications, aviation industry applications, etc.

The hearing protection systems may include an earplug displaceable between an open position and a closed position. For example, the earplug may be displaced from the open position, where the earplug is positioned away from an ear of a user to allow the user to hear ambient sound at an unchanged volume, to the closed position, where the earplug is positioned proximal to the ear of the user to at least partly block the ambient sound and allow the user to hear the ambient sound at a reduced volume that is less than the unchanged volume. A reduced volume as used herein comprises a noise reduction rating (NRR) of at least about 10 decibels to at most about 35 decibels. In the example where the hearing protection system includes a trigger mechanism employing a microphone and a microcontroller to continually respond to a voice input of the hunter, the trigger mechanism may cause the earplug to be displaced between the open position and the closed position based at least partly on a predetermined keyword. For example, the trigger mechanism may employ a microphone connected (e.g., wire connected or wirelessly connected) with one or more speech recognition components. The microphone to receive a voice input of the hunter and the one or more speech recognition components to determine if a predetermined keyword was received by the microphone. If the predetermined keyword was received, the microcontroller causes the earplug to be displaced between the open position and the closed position.

Further, the hearing protection system may include an earplug attachable proximate to an ear of a user. For example, the hearing protection system may include a receptacle receivable by the ear of the user, and the earplug may be displaceably attached to the receptacle. The earplug may be displaceably attached to the receptacle such that when in the closed position the earplug is removeably received by a cavity of the receptacle to at least partly seal the cavity of the receptacle and to at least partly block ambient sound and allow the user to hear the ambient sound at a reduced volume.

The hearing protection systems may include a servomechanism coupled to the earplug. The servomechanism being coupled to the earplug to displace the earplug between the open position and the closed position based at least in part on a signal received from the trigger mechanism. For example, the servomechanism may receive a wired or a wireless signal from the trigger mechanism that causes the servomechanism to displace the earplug between the open position and the closed portion.

### Illustrative Hearing Protection Systems

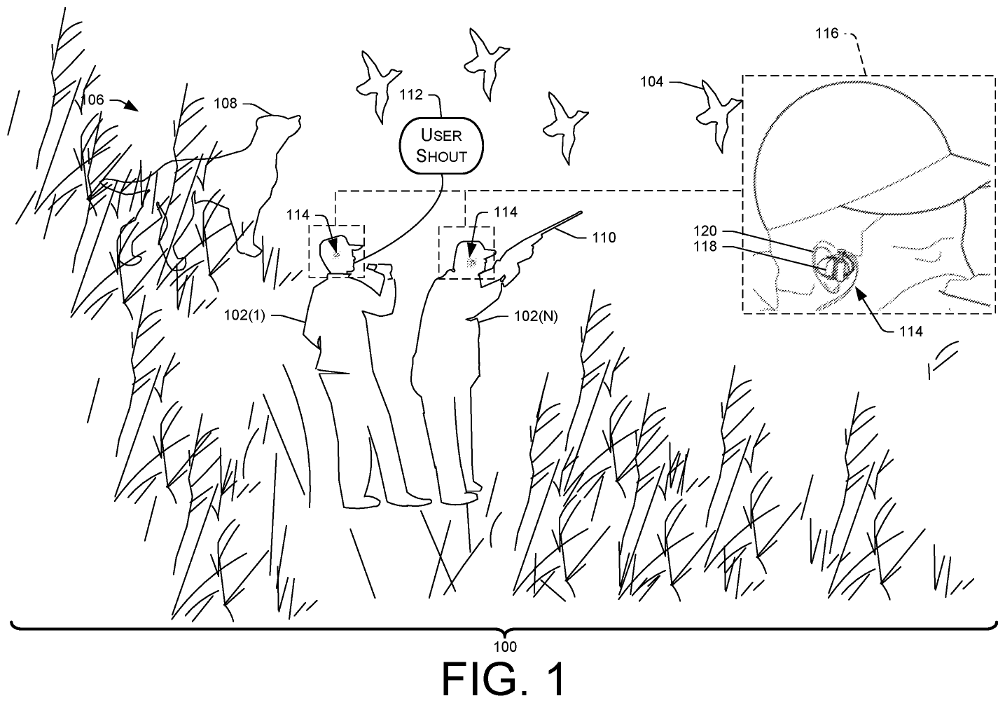

FIG. 1 illustrates an example hunting environment 100 involving hunters 102(1) and 102(N). For example, FIG. 1 illustrates a hunting environment 100 of the hunters 102(1) and 102(N) attempting to shoot game 104 flushed out of tall grass 106 by a dog 108. Further, while attempting to shoot the game 104 with a firearm 110, the hunters 102(1) and 102(N) may voice a predetermined keyword 112 “rooster,” “hen,” “open,” “close,” “shoot,” “fire,” etc. with regard to the game 104. For example, the hunter 102(1) may voice the predetermined keyword 112 “rooster” indicating the game 104 is a male bird and the hunters 102(1) and 102(N) are to proceed with attempting to shoot the game 104. As result of the predetermined keyword 112 “rooster,” a hearing protection system 114 used by the hunters 102(1) and 102(N) is displaced from an open position to a closed position to protect the ears of the hunters 102(1) and 102(N) from the noise produced from firing the firearm 110. In another example, the hunter 102(1) may voice the predetermined keyword 112 “hen” indicating the game 104 is a female bird and the hunters 102(1) and 102(N) are to refrain from attempting to shoot game 104. As result of the predetermined keyword 112 “hen,” the hearing protection system 114 used by the hunters 102(1) and 102(N) is not displaced from an open position to a closed position to allow the ears of the hunters 102(1) and 102(N) to hear ambient sound naturally at an unchanged volume.

In another example, the hunter 102(1) may voice the predetermined keyword 112 “open” indicating the hunters 102(1) and 102(N) are not attempting to shoot the game 104. As result of the predetermined keyword 112 “open,” a hearing protection system 114 used by the hunters 102(1) and 102(N) are displaced from a closed position to an open position to allow the ears of the hunters 102(1) and 102(N) to hear ambient sound naturally at an unchanged volume. As used herein, a predetermined keyword comprises a word established in advance of utilizing the hearing protection systems. For example, a user may establish a keyword via a keyword selection module that allows the user to choose a desired keyword for use with a trigger mechanism responsive to a voice input. In another example, a keyword may be pre-established in a trigger mechanism. For example, a keyword may be programmed in memory of a microcontroller for use with a trigger mechanism.

While FIG. 1 illustrates a hunting environment 100 where the hunters 102(1) and 102(N) may voice a predetermined keyword 112 to cause the hearing protection system 114 to displace between an open position and a closed position, other types of hunting environments may occur. For example, a hunting environment may occur where the firearm is positioned to fire at the game relative to a portion of the body of the hunter. As a result of the firearm being positioned to fire at the game, the hearing protection system used by the hunters may be displaced from an open position to a closed position to protect the ears of the hunters from the noise produced from firing the firearm, as described in U.S. Pat. No. 10,130,517, which is incorporated by reference herein in its entirety. The hearing protection system 114 may utilize both types of trigger mechanisms. For example, the hearing protection system 114 may utilize the trigger mechanism, responsive to a voice input, to cause the earplug to displace between the open position and the closed position based at least in part on receiving a predetermined keyword, and/or utilize the trigger mechanism that causes the earplug to displace between the open position and the closed position based at least in part on a position of a device as described in U.S. Pat. No. 10,130,517.

Detail view 116 illustrates the hearing protection system 114 in more detail and shows an earplug 118 of the hearing protection system 114 arranged with the ears 120 of the hunters 102(1) and 102(N). Detail view 116 illustrates the earplug 118 of the hearing protection system 114 arranged to protect the hearing of the hunters 102(1) and 102(N) from the noise produced from firing the firearm 110 at the game 104. For example, detail view 116 illustrates the earplug 118 of the hearing protection system 114 displaced from the open position to the closed position as a result of one of the hunters 102(1) and 102(N) voicing the predetermined keyword 112 “rooster” to fire at the game 104.

When the hearing protection system 114 is in the open position, the earplugs 118 are positioned away from the ears 120 of the hunters 102(1) and 102(N) to allow the hunters 102(1) and 102(N) to hear ambient sound at an unchanged volume. For example, when in the open position, the earplugs 118 are positioned away from the ears 120 of the hunters 102(1) and 102(N) to allow the hunters 102(1) and 102(N) to hear, naturally in the hunting environment 100, the dog 108 moving in the tall grass 106, the game 104 being flushed out of the tall grass 106, and verbal communication between the hunters 102(1) and 102(N) until the hearing protection system 114 responds to one of the hunters 102(1) and 102(N) voicing the predetermined keyword 112. Because the earplugs 118 are positioned away from the ears 120 of the hunters 102(1) and 102(N), until one of the hunters 102(1) and 102(N) voices the predetermined keyword 112, the hearing protection system 114 allows the hunters 102(1) and 102(N) to hear the ambient sound naturally at an unchanged volume making the hearing protection system 114 more efficient and safe to use than earplugs that block the ambient sound, and more efficient and safe to use than noise cancelling earplugs that inject reproduced ambient sound into the ears. Moreover, because the earplugs 118 are positioned away from the ears 120 of the hunters 102(1) and 102(N) until the voicing of the predetermined keyword 112, the earplugs 118 allow heat to dissipate from the ears 120, and thus do not cause the ears 120 to perspire or the hunters 102(1) and 102(N) to overheat, making the hearing protection system 114 more comfortable to use than the earplugs and the noise cancelling earplugs that remain stuck in the ears of a user even when the earplugs and the noise cancelling earplugs are not needed.

When one of the hunters 102(1) and 102(N) voices the predetermined keyword 112 to fire at the game 104, the hearing protection system 114 transitions to the closed position and the earplugs 118 are positioned proximal to the ears 120 of the hunters 102(1) and 102(N) to at least partly block the ambient sound and allow for the hunters 102(1) and 102(N) to hear the ambient sound at a reduced volume (e.g., a noise reduction of at least about 10 decibels to at most about 35 decibels) that is less than the unchanged volume. For example, when one of the hunters 102(1) and 102(N) voices the predetermined keyword 112 to fire at the game 104, the earplugs 118 may be positioned proximal to the ears 120 of the hunters 102(1) and 102(N) to reduce the firing noise of the firearm received by the ears 120 of the hunters 102(1) and 102(N) by at least about 10 decibels to at most about 35 decibels to protect the hearing of the ears 120 of the hunters 102(1) and 102(N).

While FIG. 1 illustrates the hearing protection systems 114 arranged in the ears 120 of the hunters 102(1) and 102(N), the hearing protection systems 114 may not be arranged in the ears 120. For example, the hearing protection system 114 may be arranged with a headband, a muff, a helmet, a hat, a cap, a stocking cap, eyewear, etc. arranged to fit on the head of the user and interface with the ears of the user. Moreover, while FIG. 1 illustrates the hearing protection systems 114 including earplugs 118 arranged to fit in the ears 120 of the hunters 102(1) and 102(N), the hearing protection system 114 may include ear muffs, ear cups, ear pads, etc. arranged to fit on and/or over the ears 120 of the user.

The hearing protection system 114 may use a trigger mechanism, responsive to a voice input, to cause the earplugs 118 to displace between the open position and the closed position based at least in part on receiving the predetermined keyword 112. For example, the trigger mechanism may include a microphone and a microcontroller arranged with the hearing protection system 114, and the trigger mechanism, responsive to receiving the voice input, causes the earplug 118 to displace between the open position and the closed position based at least in part on the predetermined keyword 112. For example, a microphone and one or more speech recognition components may be disposed with the hearing protection system that determines a presence of the predetermined keyword 112 in response to receiving the voice input. Depending on the predetermined keyword 112 present in the voice input, the trigger mechanism may displace the earplugs 118 from the open position to the closed position, displace the earplugs 118 from the closed position to the open position, keep the earplugs 118 in the closed position, keep the earplugs in the open position, etc.

FIG. 1 illustrates the hearing protection systems 114 may be connected to one or more hunters 102(1) and 102(N). For example, the hearing protection systems 114 of the hunters 102(1) and 102(N) may be communicatively coupled via wireless interconnection. For example, the wireless interconnection interconnecting the hearing protection systems 114 of the hunters 102(1) and 102(N) may be a radio interconnection, an infrared interconnection, a Bluetooth interconnection, etc. In an example where the hearing protection system 114 of the hunter 102(N) is wirelessly interconnected to the hearing protection system 114 of the hunter 102(1), the hearing protection system 114 of the hunter 102(N) may signal the hearing protection system 114 of the hunter 102(1) to displace from the open position to the closed position to protect the hearing of the hunter 102(1) from the noise produced from the hunter 102(N) firing the firearm 110. For example, the hearing protection systems 114 may include a transmitter, transceiver, antenna, etc. arranged with the trigger mechanisms of the hearing protection systems 114. The trigger mechanism of the hearing protection system 114 of the hunter 102(1) may send a signal to the hearing protection system 114 of the hunter 102(N). When the trigger mechanism of the hearing protection system 114 of the hunter 102(1) responds to the voice input, and causes the earplugs 118 of the hunter 102(1) to displace between the open position and the closed position based at least in part receiving the predetermined keyword 112, the hearing protection system 114 of the hunter 102(1) may send a signal to the trigger mechanism of the hearing protection system 114 of the hunter 102(N) to displace the earplugs 118 of the hunter 102(N) between the open position and the closed position.

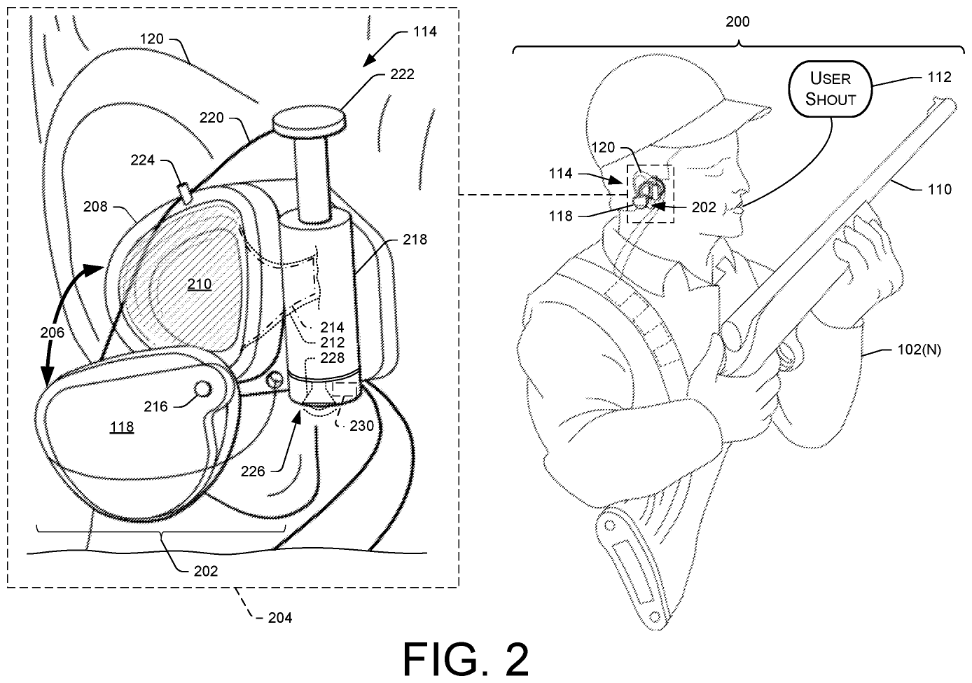

FIG. 2 illustrates a perspective view 200 of the example hearing protection system 114 shown in FIG. 1 in an open position 202 that allows the hunters 102(1) and 102(N) shown in FIG. 1 to hear ambient sound at an unchanged volume.

Detail view 204 illustrates the hearing protection system 114 in the open position 202 in more detail and shows the earplug 118 positioned a distance 206 away from the ear 120 of the hunter 102(N) to allow the hunter 102(N) to hear ambient sound at an unchanged volume. The distance 206 the earplug 118 may be positioned away from the ear 120 may be at least about 0.06 inches (0.2 centimeters) to at most about 5 inches (13 centimeters).

Detail view 204 shows the hearing protection system 114 may include a receptacle 208. The receptacle 208 may be receivable by the ear 120. For example, the receptacle 208 may be a hollow earplug or hollow earpiece that may be received by the ear 120 of a user. The receptacle 208 may have a wall thickness of at least about 0.005 inches (0.01 centimeters) to at most about 0.02 inches (0.05 centimeters). The receptacle 208 may be formed of plastic, rubber, metal, wood, carbon fiber, fiberglass, glass, composite, etc. The receptacle 208 may include a cavity 210 arranged to fit in at least a portion of the ear 120. For example, the cavity 210 may be arranged in the receptacle 208 such that the cavity 210 interfaces with the inside the ear 120 proximate to the opening of the canal 212 (e.g., auditory canal) (illustrated as a hidden dashed line) of the ear 120. The receptacle 208 may include a channel 214 communicatively coupled to the cavity 210 and arranged to fit in at least a portion of the canal 212 of the ear 120.

Detail view 204 shows the earplug 118 is displaceably attached, via a hinge 216, to the receptacle 208. While detail view 204 shows the hinge 216 is rotatably attaching the earplug 118 to the receptacle 208, the hinge 216 may foldably, pivotably, slideably, etc. attach the earplug 118 to the receptacle 208. While detail view 204 shows the earplug 118 displaceably attached, via the hinge 216, to the receptacle 208, the earplug 118 may not be displaceably attached via the hinge 216. For example, the earplug 118 may be displaceably attached to the receptacle 208 via a hydraulic actuator, pneumatic actuator, magnetic actuator, etc. Further, while FIG. 2 illustrates the earplug 118 displaceably attached to the receptacle 208, the earplug 118 may not be displaceably attached to the receptacle 208. For example, the earplug 118 may be displaceably attached to a headband, a muff, a helmet, a hat, a cap, a stocking cap, eyewear, etc. arranged to fit on the head of the user.

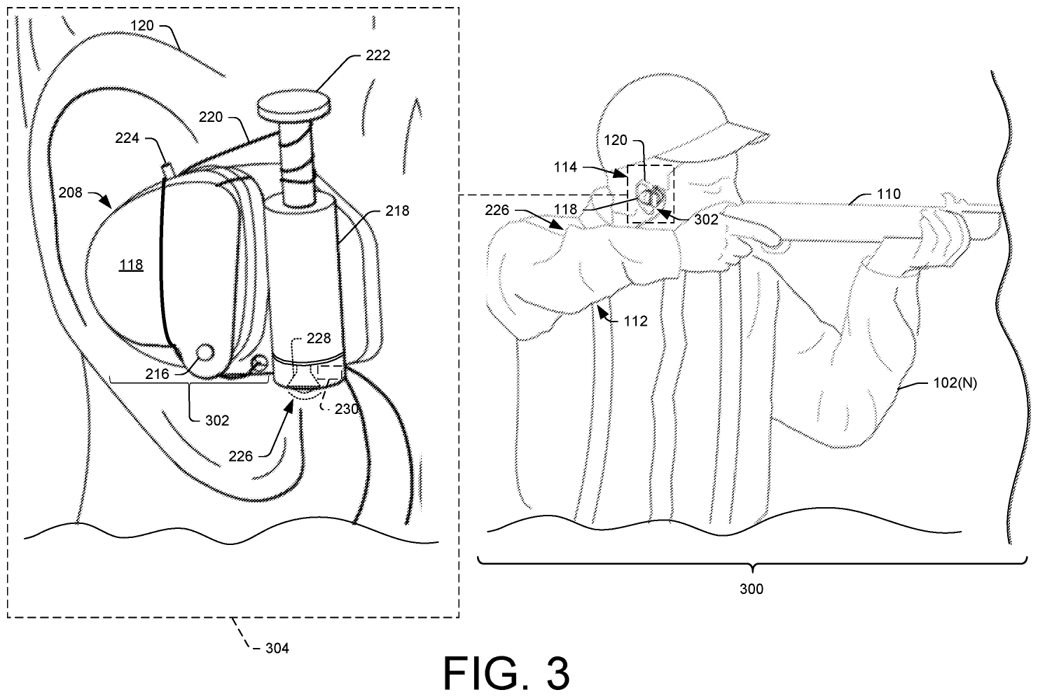

Detail view 204 shows a motor 218 coupled to the earplug 118. For example, the motor 218 may be fixed to the receptacle 208 and coupled, via a member 220, to the earplug 118. The member 220 may be a line, wire, a chain, etc. having first end fixed to the earplug 118 and second end fixed to a rotating shaft 222 of the motor 218. When the motor 218 rotates the rotating shaft 222, the member 220 wraps around the rotating shaft 222. As the member 220 wraps around the rotating shaft 222, the member 220 displaces the earplug 118 from the open position 202 to the closed position (described in detail with regard to FIG. 3 below). Detail view 204 shows a guide member 224 fixed to the receptacle 208 and arranged to guide the member 220 as the member 220 is displaced via the rotating shaft 222. The guide member 224 may be a post, a hook, a ring, a ferrule, etc. arranged to guide the member 220 as the member 220 is displaced via the rotating shaft 222.

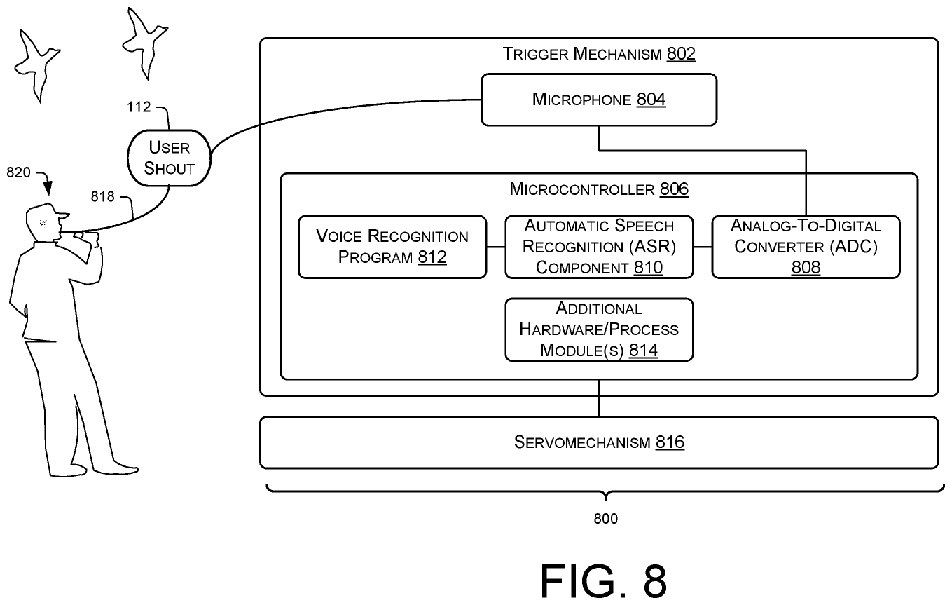

FIG. 2 illustrates a trigger mechanism 226 may be disposed with the motor 218. Trigger mechanism 226 may include a microphone 228 for receiving the voice input from at least one of the hunters 102(1) and 102(N). The trigger mechanism 226 may include a microcontroller 230 connected to the microphone 228. The microcontroller 230 may include one or more speech recognition components (e.g., a voice recognition program, an analog-to-digital converter (ADC), and an automatic speech recognition (ASR) component, etc.). The microcontroller 230 to determine if the predetermined keyword 112 is present in the voice input received from the microphone 228 (discussed in more detail below with regard to FIGS. 8 through 11). The microcontroller 230 controls the motor 218 that displaces the earplugs 118 from the open position 202 to the closed position. While FIG. 2 illustrates the trigger mechanism 226 disposed with the motor 218, the trigger mechanism 226 may be disposed with the receptacle 208 or the earplug 118 of the hearing protection system 114. Further, the trigger mechanism 226 may be disposed with a portion of the user. While FIG. 2 illustrates the trigger mechanism 226 disposed with the motor 218, in an alternative embodiment, the trigger mechanism 226 may not be disposed with the motor 218. For example, the trigger mechanism 226 may be a stand-alone or self-contained system. For example, the trigger mechanism 226 may be a separate electronic device that includes the microphone 228 and/or the microcontroller 230. The separate electronic device may be a wearable electronic device. For example, the separate electronic device may be a neckband device (e.g., neckband headphone device) wearable on a neck of a user, a wristband device (e.g., smartwatch device) wearable on a wrist of a user, an armband device wearable on an arm of a user, an eye protection device (e.g., smart glasses device, augmented reality glasses device) wearable on a face of a user, etc. The wearable electronic device may be a wireless device. For example, the wearable electronic device may be communicatively coupled via wireless interconnection to the hearing protection system 114. The wireless interconnection interconnecting the wearable electronic device and the hearing protection system 114 may be a radio interconnection, an infrared interconnection, a Bluetooth interconnection, etc.

FIG. 3 illustrates a perspective view 300 of the example hearing protection system 114 shown in FIG. 2 in a closed position 302 that allows the hunters 102(1) and 102(N) shown in FIG. 1 to hear ambient sound at a reduced volume that is less than the unchanged volume.

Detail view 304 illustrates the hearing protection system 114 in the closed position 302 in more detail and shows the earplug 118 is positioned proximal to the ear 120 of the hunter 102(N) to at least partly block the ambient sound and allow the hunter 102(N) to hear the ambient sound at a reduced volume that is less than the unchanged volume. For example, the earplug 118 may be positioned in the ear 120 to at least partly block the ambient sound and allow the hunter 102(N) to hear the ambient sound at a reduced volume that is less than the unchanged volume. For example, when the earplug 118 is in the closed position 302, the earplug 118 may be removeably received by the cavity 210 of the receptacle 208 to at least partly seal the cavity 210 of the receptacle 208 and at least partly block the ambient sound and allow the hunter 102(N) to hear the ambient sound at the reduced volume. In another example, when the earplug 118 is in the closed position 302, the earplug 118 may be removeably received by the cavity 210 of the receptacle 208 to at least partly seal the channel 214 of the receptacle 208 and at least partly block the ambient sound and allow the user to hear the ambient sound at the reduced volume. The earplug 118 may be a cap, a flap, a plug, etc. arranged to cover the receptacle 208, or the cavity 210 of the receptacle 208. Moreover, the earplug 118 may be arranged to fit on or over the ear 120. For example, the earplug 118 may be an ear muff, an ear cup, an ear pad, etc. arranged to fit on or over the ear 120.

FIG. 3 illustrates the trigger mechanism 226 having caused the earplug 118 to displace from the open position 202 to the closed position 302 based at least in part on receiving the predetermined keyword 112. For example, the hunter 102(N) may have voiced (e.g., shouted) the predetermined keyword 112 “rooster” indicating the game 104 is a male bird and the hunter 102(N) is going to proceed with attempting to shoot the game 104. The microphone 228 receives the voiced input, and the microcontroller 230 determines whether the predetermined keyword 112 is present in the voiced input received by the microphone 228. As result of the microcontroller 230 determining the predetermined keyword 112 “rooster” is present in the voiced input received by the microphone 228, the hearing earplug 118 is displaced from the open position illustrated in FIG. 2 to the closed position illustrated in FIG. 3 to protect the ears of the hunters 102(1) and 102(N) from the noise produced from firing the firearm 110. For example, FIG. 3 illustrates microcontroller 230 having recognized the predetermined keyword 112 and having controlled the motor 218 that displaced the earplug 118 from the open position 202 to the closed position 302.

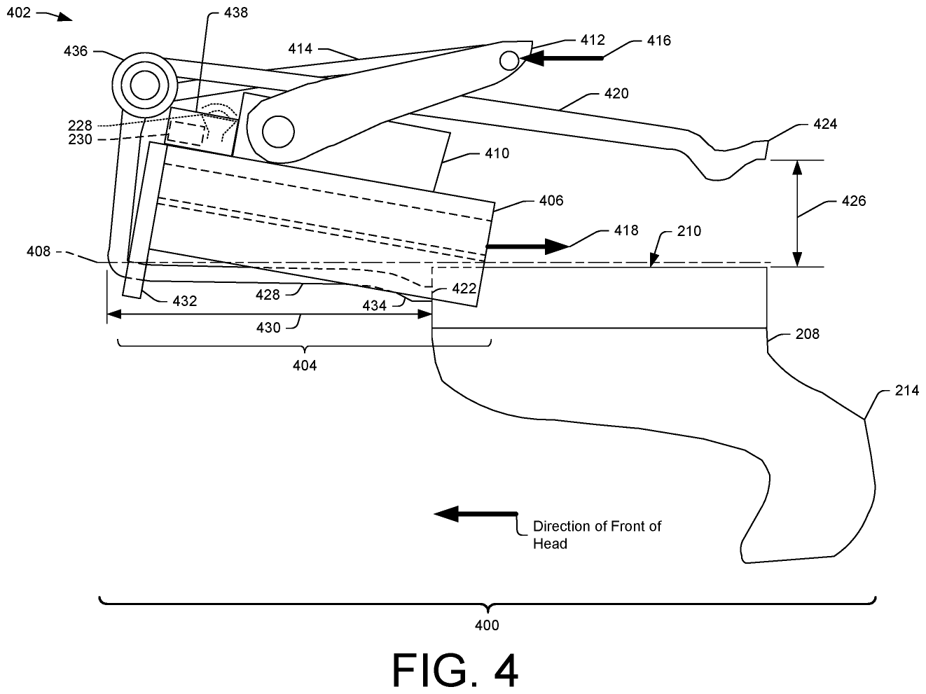

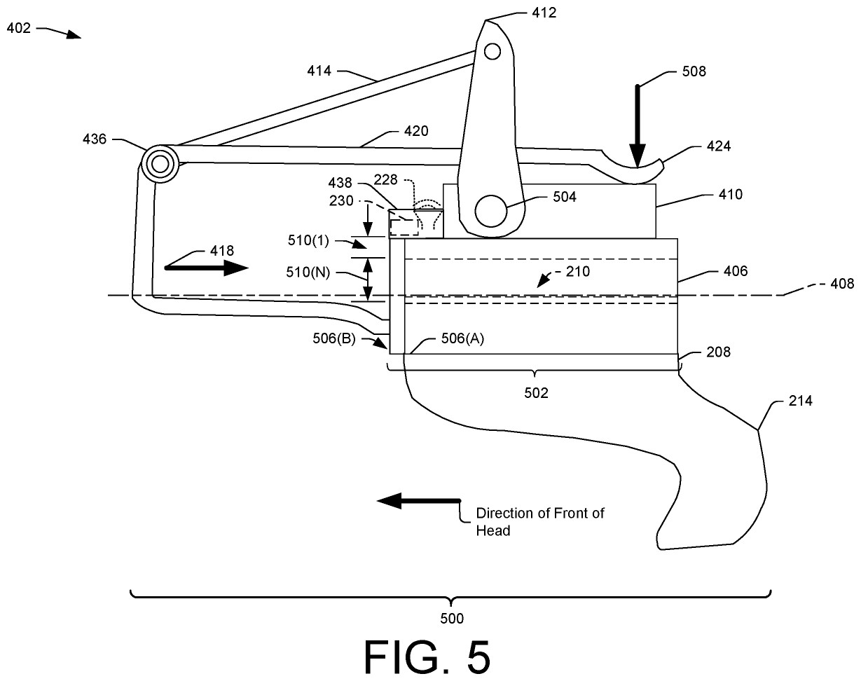

FIG. 4 illustrates a side view 400 of another example hearing protection system 402 in an open position 404 that allows the users shown in FIG. 1 to hear ambient sound at an unchanged volume, and FIG. 5 illustrates a side view 500 of the hearing protection system 402 shown in FIG. 4 in a closed position 502 that allows the hunters shown in FIG. 1 to hear ambient sound at a reduced volume that is less than the unchanged volume. Inasmuch as FIGS. 4 and 5 depict the hearing protection system 402 in the open position 404 and the closed position 502, while referring to the same elements and features of the hearing protection system 402, the following discussion of specific features may refer interchangeably to any of FIGS. 1, 2, 3, 4, and 5 except where explicitly indicated. In particular, FIGS. 4 and 5 illustrate an embodiment of the hearing protection system 402, including the receptacle 208. The receptacle 208 may be receivable by the ear 120 (illustrated in FIGS. 1, 2, and 3) and may include the cavity 210 arranged to fit in at least a portion of the ear 120. The receptacle 208 may include the channel 214 communicatively coupled to the cavity 210 and arranged to fit in at least a portion of the canal 212 of the ear 120.

Similar to the hearing protection system 114 of FIGS. 1, 2, and 3, the hearing protection system 402 is configured to protect a hearing of users (e.g., hunters 102(1) and 102(N)) from noise produced from firing a firearm (e.g., firearm 110). The actuation of the hearing protection system 402 may cause an earplug 406 of the hearing protection system 402 to be displaced from the open position 404 to the closed position 502 to protect the hearing ability of users from the noise produced from firing the firearm.

FIGS. 4 and 5 depict the earplug 406 of the hearing protection system 402 may be moveably disposed in a plane 408 covering the cavity 210 of the receptacle 208. The earplug 406 may moveably displace in the plane 408 between the open position 404 and the closed position 502. FIG. 4 illustrates that when the earplug 406 is in the open position 404, the earplug 406 is positioned adjacent to the cavity 210 of the receptacle 208 to allow the user to hear ambient sound at an unchanged volume. FIG. 5 illustrates that when the earplug 406 is in the closed position 502, the earplug 406 is positioned over the cavity 210 of the receptacle 208 to at least partly block the ambient sound and allow the user to hear the ambient sound at a reduced volume that is less than the unchanged volume.

FIG. 4 depicts the hearing protection system 402 may include a servomechanism 410. The servomechanism 410 may be attached to the earplug 406. In one example, the servomechanism 410 may be attached to a top surface of the earplug 406. In another example, the servomechanism 410 may be attached to a side surface of the earplug 406. In another example, the servomechanism 410 may be attached to an inside surface of the earplug 406. In another example, the servomechanism 410 may be attached to the receptacle 208.

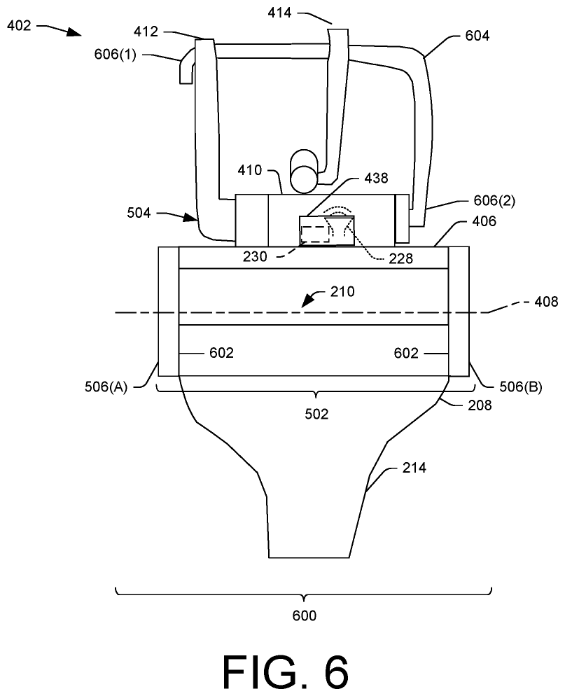

An arm 412 of the servomechanism 410 may be pivotably attached to a link 414. For example, the arm 412 of the servomechanism 410 may be pivotably attached to the link 414 via a bail or torque bail (explained in more detail below with regard to FIG. 6). The arm 412 of the servomechanism 410 may apply a force in a direction 416 to a portion of the link 414 to cause the earplug 406 to be displaced in a direction 418, from the open position 404, to the closed position 502 when the servomechanism 410 is actuated.

FIG. 4 depicts the link 414 may be pivotably attached to a structural member 420. The structural member 420 may attach to a portion of the receptacle 208. The structural member 420 may be an elongated member (e.g., a wire, a spring wire, a rod, etc.) attached to the portion of the receptacle 208. For example, the structural member 420 may be a spring wire having a first end 422 attached to the receptacle 208 and a second end 424 arranged a distance 426 above the receptacle 208. The structural member 420 may include a guide portion 428 extending a distance 430 from the receptacle 208. The guide portion 428 of the structural member 420 may provide a guide (e.g., a rail, a beam, a support, etc.) for the earplug 406 when the earplug 406 is moveably displaced. For example, the earplug 406 may include a follower 432 attached to the earplug 406 that may be guided by the guide portion 428 of the structural member 420 when the earplug 406 is moveably displaced. For example, the follower 432 may be linearly guided along the guide portion 428 of the structural member such that the earplug 406 is linearly displaced in the plane 408 covering the cavity 210 of the receptacle 208. The guide portion 428 may have a bend 434 arranged proximate to the receptacle 208. The bend 434 may provide for the guide portion 428 to guide the earplug 406 to a position on a surface of the receptacle 208 as the earplug 406 is displaced the direction 418 from the open position 404 to the closed position 502 when the servomechanism 410 is actuated.

FIG. 4 depicts the structural member 420 may include an attachment portion 436 arranged a distance from the guide portion 428. The attachment portion 436 may provide for pivotably attaching an end of the link 414 opposite the portion of the link 414 pivotably attached to the arm 412 of the servomechanism 410 via the bail or torque bail. The attachment portion 436 of the structural member 420 may comprise an opening formed in the structural member 420. For example, the structural member 420 may be an elongated member (e.g., a wire, a spring wire, a rod, etc.) and the opening may be formed of a winded portion (e.g., a coil, a twist, a loop, etc.) of the elongated member. The winding may be formed of about a 450 degrees winding of the elongated member. The winding of the structural member 420 may also provide at least an amount of spring force to the second end 424 of the structural member 420 for applying a force in a direction orthogonal to the plane 408 to force the earplug 406 onto the receptacle 208 and at least partially close or at least partially seal (e.g., air tight seal) the cavity 210 of the receptacle 208 (explained in more detail below with regard to FIG. 5).

Similar to the hearing protection system 114, the hearing protection system 402 may use a trigger mechanism 438, responsive to a voice input, to cause the earplug 406 to displace between the open position 404 and the closed position 502 based at least in part on receiving a predetermined keyword 112. For example, the trigger mechanism 438 may include the microphone 228 for receiving the voice input from at least one of the hunters 102(1) and 102(N) and the microcontroller 230 that controls the servomechanism 410 that displaces the earplug 406 from the open position 404 to the closed position 502. While FIG. 2 illustrates the trigger mechanism 438 disposed with the earplug 406, the trigger mechanism 438 may be disposed with the servomechanism 410, or the receptacle 208 of the hearing protection system 402. Further, the trigger mechanism 438 may be disposed with a portion of a user.

FIG. 5 depicts the hearing protection system 402 in the closed position 502 where the earplug 406 is positioned over the cavity 210 of the receptacle 208 to at least partly block the ambient sound and allow the user to hear the ambient sound at a reduced volume that is less than the unchanged volume. FIG. 5 depicts the arm 412 of the servomechanism 410 may rotate counterclockwise relative to a shaft 504 of the servomechanism 410 to cause the earplug 406 to be displaced in the direction 418 to the closed position 502. To displace the earplug 406 to the open position 404 illustrated in FIG. 4, the arm 412 of the servomechanism 410 may rotate clockwise relative to the shaft 504 of the servomechanism 410 to cause the earplug 406 to be displaced in the opposite direction of the direction 418 to the open position 404.

FIG. 5 depicts the earplug 406 may include one or more guide rails 506(A) and 506(B). The one or more guide rails 506(A) and 506(B) (also shown in FIG. 6) may provide for guiding the earplug 406 over the receptacle 208. For example, the one or more guide rails 506(A) and 506(B) may cooperate with an outside surface of the receptacle 208 to slideably guide the earplug 406 linearly over the receptacle 208 such that the earplug 406 is linearly displaced in the plane 408 covering the cavity 210 of the receptacle 208.

Further, FIG. 5 depicts the second end 424 of the structural member 420 may be shifted from a free position to a contact position against the servomechanism 410 as the servomechanism 410 is displaced under the second end 424 of the structural member 420. For example, as the arm 412 of the servomechanism 410 rotates counterclockwise to cause the earplug 406 to be displaced in the direction 418 to the closed position 502, the second end 424 of the structural member 420 may be displaced from the free position by the sliding insertion of the servomechanism 410 between the structural member 420 and the receptacle 208. The second end 424 of the structural member 420 may apply a force in a direction 508 orthogonal to the plane 408 covering the cavity 210 of the receptacle 208 to at least partially close or at least partially seal (e.g., air tight seal) the cavity 210 of the receptacle 208 and at least partly block the ambient sound and allow the user to hear the ambient sound at the reduced volume. The attachment portion 436 of the structural member 420 may provide at least an amount of spring force or biasing force to the second end 424 of the structural member 420. For example, the attachment portion 436 may be formed of a winding (e.g., a coil, a twist, a loop, etc.) that may provide at least an amount of spring force to the second end 424 of the structural member 420. The winding of the attachment portion 436 may provide for applying the force in the direction 508 orthogonal to the plane 408. The force applied in the direction 508 may force the earplug 406 onto the receptacle 208 and at least partially close or at least partially seal (e.g., air tight seal) the cavity 210 of the receptacle 208 and at least partly block the ambient sound and allow the user to hear the ambient sound at the reduced volume (explained in more detail below with regard to FIG. 5). Further, the second end 424 of the structural member 420 may apply the force in the direction 508 orthogonal to the plane 408 covering the cavity 210 of the receptacle 208 as a result of the second end 424 of the structural member 420 being displaced a distance greater than the distance 426. For example, when the second end 424 of the structural member 420 is displaced a distance greater than the distance 426, the winding or spring of the attachment portion 436 may be displaced (e.g., stretched) from the springs resting position and provide at least an amount of spring force to the second end 424 of the structural member 420 to apply the force in the direction 508.

The second end 424 of the structural member 420 may have a curved portion (e.g., a hook, a bend, a bump, etc.) that may apply the force in the direction 508 orthogonal to the plane 408 covering the cavity 210 of the receptacle 208 to at least partially close or at least partially seal (e.g., air tight seal) the cavity 210 of the receptacle 208 and at least partly block the ambient sound and allow the user to hear the ambient sound at the reduced volume. In some examples, when the arm 412 of the servomechanism 410 has completed the rotation counterclockwise to cause the earplug 406 to be in the closed position 502, the arm 412 may apply a force in the direction 508 orthogonal to the plane 408 to at least partially close or at least partially seal (e.g., air tight seal) the cavity 210 of the receptacle 208 and at least partly block the ambient sound and allow the user to hear the ambient sound at the reduced volume.

FIG. 5 depicts the earplug 406 may include one or more layers 510(1) and 510(N) of material arranged adjacent to each. Each of the one or more layers 510(1) and 510(N) may be formed of a material having a different density. For example, the layer 510(1) may be formed of a first material having a first density, and the layer 510(N) may be formed of a second material having a second density different from the first density of the first material. In one example, the layer 510(1) may be formed of a composite (e.g., a wood composite, a plywood, a modeling plywood, a fiberglass, a carbon fiber reinforced polymer, etc.), and the layer 510(N) may be formed of a plastic (e.g., polyethylene (PE), polypropylene (PP), polystyrene (PS), polyamides (PA), polyurethanes (PU), silicon, etc.). In another example, the layers 510(1) and 510(N) may each be formed of a plastic.

The layer 510(1) may be formed of a material having a density greater than a density of a material forming the layer 510(N). For example, the layer 510(1) may be formed of a material having a greater density than the density of the material forming the layer 510(N) to provide for mounting the servomechanism 410 to the layer 510(1). Moreover, the layer 510(N) may be formed of a material having a lesser density than the density of the material forming the layer 510(1) to provide for at least partially sealing (e.g., air tight seal) the earplug 406 to the cavity 210 of the receptacle 208. For example, the layer 510(N) may be formed of a silicon (e.g., a low durometer silicone) to provide for at least partially sealing (e.g., air tight seal) the earplug 406 to the cavity 210 of the receptacle 208. For example, the second end 424 of the structural member 420 may apply the force in the direction 508 orthogonal to the plane 408 covering the cavity 210 of the receptacle 208 to compress the layer 510(N) formed of a low durometer material between the first layer 510(1) and the receptacle 208 to at least partially close or at least partially seal (e.g., air tight seal) the cavity 210 of the receptacle 208 and at least partly block the ambient sound and allow the user to hear the ambient sound at the reduced volume. While FIG. 5 illustrates the earplug 406 comprising two layers, the earplug 406 may comprise fewer than two layers or may comprise more than two layers. The one or more layers 510(1) and 510(N) may have a total thickness that provides for at least partly blocking the ambient sound and allow the user to hear the ambient sound at the reduced volume.

FIG. 6 illustrates a back view 600 of the hearing protection system 402 shown in FIG. 5 in the closed position 502 that allows the hunters shown in FIG. 1 to hear ambient sound at a reduced volume that is less than the unchanged volume. FIG. 6 depicts the one or more guide rails 506(A) and 506(B) as cooperating with the outside surface 602 of the receptacle 208 to slideably guide the earplug 406 linearly over the receptacle 208 such that the earplug 406 is linearly displaced in the plane 408 covering the cavity 210 of the receptacle 208.

FIG. 6 also depicts that the arm 412 of the servomechanism 410 may be pivotably attached to the link 414 via a bail 604 (e.g., torque bail). A first end 606(1) of the bail 604 may be pivotably attached to the arm 412 of the servomechanism 410 and a second end 606(2) of the bail 604 may be pivotably attached to a portion of the earplug 406. For example, the first end 606(1) of the bail 604 may be pivotably attached to an end of the arm 412 and the second end 606(2) of the bail 604 may be pivotably attached to the servomechanism 410. FIG. 6 depicts the link 414 pivotably attached to the bail 604. For example, the link 414 may be pivotably attached to a portion of the bail 604 arranged between the first end 606(1) and the second end 606(2) of the bail 604. Thus, when the servomechanism 410 is actuated by a trigger mechanism 438 to cause the earplug 406 to be displaced from the open position 404 to the closed position 502, the arm 412 of the servomechanism 410 may rotate counterclockwise relative to the shaft 504 of the servomechanism 410 and apply a force in the direction 416 to the portion of the link 414 via the bail 604 to cause the earplug 406 to be displaced from the open position 404 to the closed position 502. Further, when the servomechanism 410 is actuated by a trigger mechanism 438 to cause the earplug 406 to be displaced from the closed position 502 to the open position 404, the arm 412 of the servomechanism 410 may rotate clockwise relative to the shaft 504 of the servomechanism 410 and apply a force in a direction opposite the direction 416 to the portion of the link 414 via the bail 604 to cause the earplug 406 to be displaced from the closed position 502 to the open position 404.

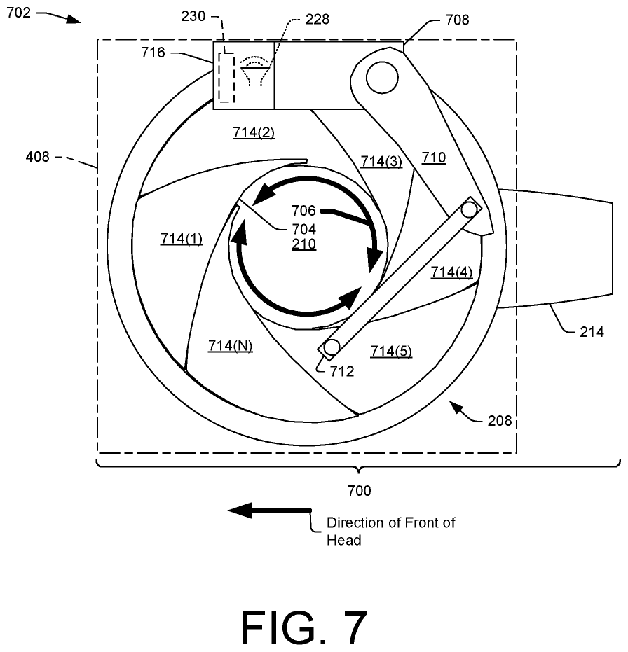

FIG. 7 illustrates a top view 700 of another example hearing protection system 702. The hearing protection systems 702 may include an earplug 704 displaceable between an open position and a closed position. For example, the earplug 704 may be displaced from the open position, where the earplug 704 is positioned such that cavity 210 is exposed to the atmosphere to allow the user to hear ambient sound at an unchanged volume, to the closed position 502, where the earplug 704 is positioned such that the cavity is closed off from the atmosphere to at least partly block the ambient sound and allow the user to hear the ambient sound at a reduced volume that is less than the unchanged volume.

Inasmuch as FIG. 7 depicts the hearing protection system 702 being displaceable between the open position 404 and the closed position 502, while referring to the same elements and features of the hearing protection system 702, the following discussion of specific features may refer interchangeably to any of FIGS. 1, 2, 3, 4, 5, and 6 except where explicitly indicated. In particular, FIG. 7 illustrates an embodiment of the hearing protection system 702, including the receptacle 208. The receptacle 208 may be receivable by the ear 120 (illustrated in FIGS. 1, 2, and 3) and may include the cavity 210 arranged to fit in at least a portion of the ear 120. The receptacle 208 may include the channel 214 communicatively coupled to the cavity 210 and arranged to fit in at least a portion of the canal 212 of the ear 120.

Similar to the hearing protection systems 114 of FIGS. 1, 2, 3, and the hearing protection systems 402 of FIGS. 4, 5, and 6, the hearing protection system 702, is configured to protect a hearing of users (e.g., hunters 102(1) and 102(N)) from noise produced from firing a firearm (e.g., firearm 110). The actuation of the hearing protection system 702 may cause the earplug 704 of the hearing protection system 702 to be displaced from the open position 404 to the closed position 502 to protect the hearing abilities of users from the noise produced from firing the firearm.

FIG. 7 depicts the earplug 704 of the hearing protection system 702 may be moveably disposed in the plane 408 covering the cavity 210 of the receptacle 208. The earplug 704 may moveably displace in the plane 408 between the open position 404 and the closed position 502. FIG. 7 illustrates that when the earplug 704 is moveably displaced in the plane 408 between the open position 404 and the closed position 502, the earplug 704 may be moveably displaced radially 706 in the plane 408 above the cavity 210 of the receptacle 208 between the open position 404 and the closed position 502.

FIG. 7 depicts the hearing protection system 702 may include a servomechanism 708. The servomechanism 708 may be attached to the earplug 704. In one example, the servomechanism 708 may be attached to a top surface of the earplug 704. In another example, the servomechanism 708 may be attached to a side surface of the earplug 704. In another example, the servomechanism 708 may be attached to the receptacle 208.

An arm 710 of the servomechanism 708 may be pivotably attached to a link 712. The arm 710 of the servomechanism 708 may apply a force in a direction to a portion of the link 712 to cause the earplug 704 to be displaced radially 706, from the open position 404, to the closed position 502 when the servomechanism 708 is actuated.

FIG. 7 depicts the earplug 704 may include one or more pivoting members 714(1), 714(2), 714(3), 714(4), 714(5), and 714(N). Similar to the hearing protection system 402, each of the one or more pivoting members 714(1)-714(N) of the earplug 704 may include one or more layers (e.g., one or more layers 510(1) and 510(N)) formed of a material having a different density. In another example, each of the one or more pivoting members 714(1)-714(N) of the earplug 704 may include a single layer of material. In one example, the one or more layers of each of the one or more pivoting members 714(1)-714(N) may have a total thickness that provides for at least partly blocking the ambient sound and allow the user to hear the ambient sound at the reduced volume. In another example, the one or more layers of each of the one or more pivoting members 714(1)-714(N) may have a thickness that is a fraction of a total thickness that provides for at least partly blocking the ambient sound and allowing the user to hear the ambient sound at the reduced volume.

Similar to the hearing protection systems 114 and 402, the hearing protection system 702 may use a trigger mechanism 716 responsive to a voice input, to cause the earplug 704 to displace between the open position 404 and the closed position 502 based at least in part on receiving a predetermined keyword 112. For example, the trigger mechanism 716 may include the microphone 228 for receiving the voice input from at least one of the hunters 102(1) and 102(N) and the microcontroller 230 that controls the servomechanism 708 that displaces the earplug 704 from the open position 404 to the closed position 502. While FIG. 2 illustrates the trigger mechanism 716 disposed with the servomechanism 708, the trigger mechanism 716 may be disposed with the receptacle 208 of the hearing protection system 702. Further, the trigger mechanism 716 may be disposed with a portion of a user).

### Illustrative Embodiment of a Schematic of a Trigger Mechanism

FIG. 8 illustrates a schematic view 800 of an example trigger mechanism 802 according to an embodiment of the instant disclosure. The trigger mechanism 802 may be the same as any one of trigger mechanism 226, trigger mechanism 438, or trigger mechanism 716. FIG. 8 illustrates the trigger mechanism 802 may include a microphone 804 and a microcontroller 806. The microphone 804 may be the same as microphone 228 and the microcontroller 806 may be the same as the microcontroller 230.

The microcontroller 806 may include one or more speech recognition components. For example, the microcontroller 806 may include an analog-to-digital converter (ADC) 808, an automatic speech recognition (ASR) component 810, and a voice recognition program 812. The microphone 804 may be connected to the analog-to-digital converter (ADC) 808. The analog-to-digital converter (ADC) 808 may be connected to the automatic speech recognition (ASR) component 810. The voice recognition program 812 may be connected to the automatic speech recognition (ASR) component 810 and/or the analog-to-digital converter (ADC) 808. The microcontroller 806 may include one or more additional hardware and/or process modules 814 such as a processor, a microprocessor, memory, etc. may be implemented to assist the functions of the microcontroller 806. The microcontroller 806 may be connected to a servomechanism 816. The servomechanism 816 may be the same as the servomechanism 410 or the servomechanism 708. While FIG. 8 illustrates the microcontroller 806 connected to a servomechanism 816, the microcontroller 806 may be connected to a motor. For example, the microcontroller 806 may be connected to the motor 218 illustrated in FIG. 2.

The microphone 804 may be wire connected or wirelessly connected to the analog-to-digital converter (ADC) 808. The microcontroller 806 may be wire connected or wirelessly connected to the servomechanism 816. The microphone 804 receives a voice input 818 from a hunter 820 and produces analog sound waves representing the voice input 818. The analog-to-digital converter (ADC) 808 converts the analog sound waves received by the microphone 804 to digital data. The automatic speech recognition (ASR) component 810 analyzes the digital data received from the analog-to-digital converter (ADC) 808 to recognize the predetermined keyword 112.

The voice recognition program 812 may receive the predetermined keyword 112 from the automatic speech recognition (ASR) component 810. Based on the predetermined keyword 112 received from the automatic speech recognition (ASR) component 810, the voice recognition program 812 may control the servomechanism 816 that moves an earplug (e.g., earplug 118, earplug 406, or earplug 704) between an open position (e.g., open position 202 or open position 404) and a closed position (e.g., closed position 302 or closed position 502).

### Illustrative Embodiments of Methods of Controlling a Servomechanism

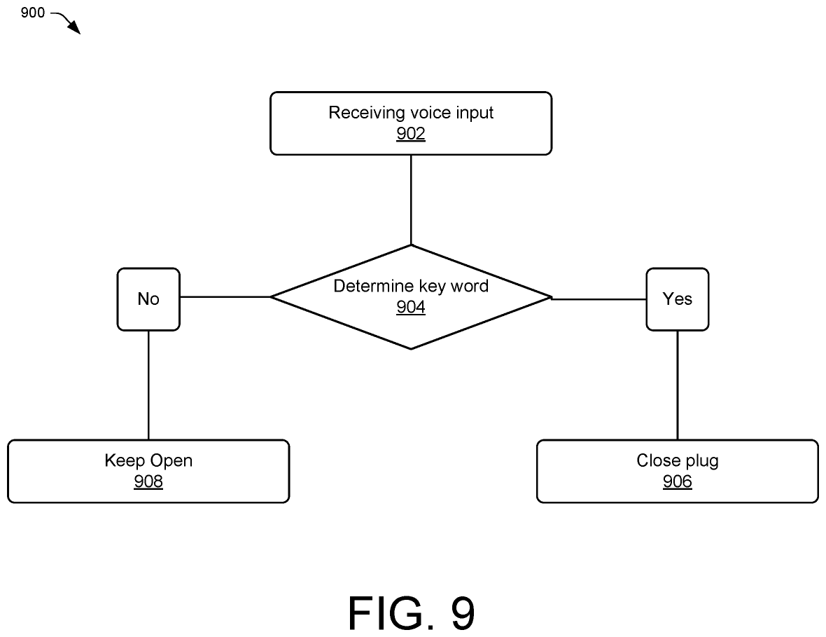

FIG. 9, illustrates an example method 900 of displacing an earplug (e.g., earplug 118, earplug 406, or earplug 704) between an open position (e.g., open position 202 or open position 404) and a closed position (e.g., closed position 302 or closed position 502) according to an embodiment of the instant disclosure. This process may be performed to cause a trigger mechanism (e.g., trigger mechanism 802) to move the earplug between the open position and the closed position based at least in part on receiving a predetermined keyword (e.g., predetermined keyword 112).

Method 900 may include operation 902, which represents receiving, by a microphone (e.g., microphone 804), a voice input (e.g., voice input 818) from a hunter (e.g., hunter 820). Operation 902 may further represent the microphone 804 producing analog sound waves representing the voice inputs.

Method 900 may proceed with operation 904, which represents determining, by a microcontroller (e.g., microcontroller 806), if a predetermined keyword (e.g., predetermined keyword 112) is present in the voice input from the hunter. For example, an analog-to-digital converter (ADC) (e.g., analog-to-digital converter (ADC) 808) may convert the analog sound waves received by the microphone to digital data. An automatic speech recognition (ASR) component (e.g., automatic speech recognition (ASR) component 810) may analyze the digital data received from the analog-to-digital converter (ADC) to recognize if the predetermined keyword is present. Method 900 may proceed to operation 906, if the predetermined keyword is present. For example, a voice recognition program (e.g., voice recognition program 812) may receive the predetermined keyword from the automatic speech recognition (ASR) component and cause a servomechanism (e.g., servomechanism 816) to displace the earplug from the open position to the closed position. Alternatively, method 900 may proceed to operation 908, if the predetermined keyword is not present. For example, the voice recognition program 814 may not receive the predetermined keyword from the automatic speech recognition (ASR) component and cause the servomechanism to refrain from displacing the earplug from the open position to the closed position and remain in the open position. In one embodiment, operation 906 may include causing the earplug to remain in the closed position based at least in part on a predetermined amount of time. In another embodiment, operation 906 includes causing the earplug to remain in the closed position based at least in part on the voice recognition program 814 receiving another predetermined keyword (e.g., “open”). For example, the trigger mechanism may be further responsive to a second voice input, to cause the earplug to displace from the closed position to the open position based at least in part on receiving a second predetermined keyword (e.g., “open”).

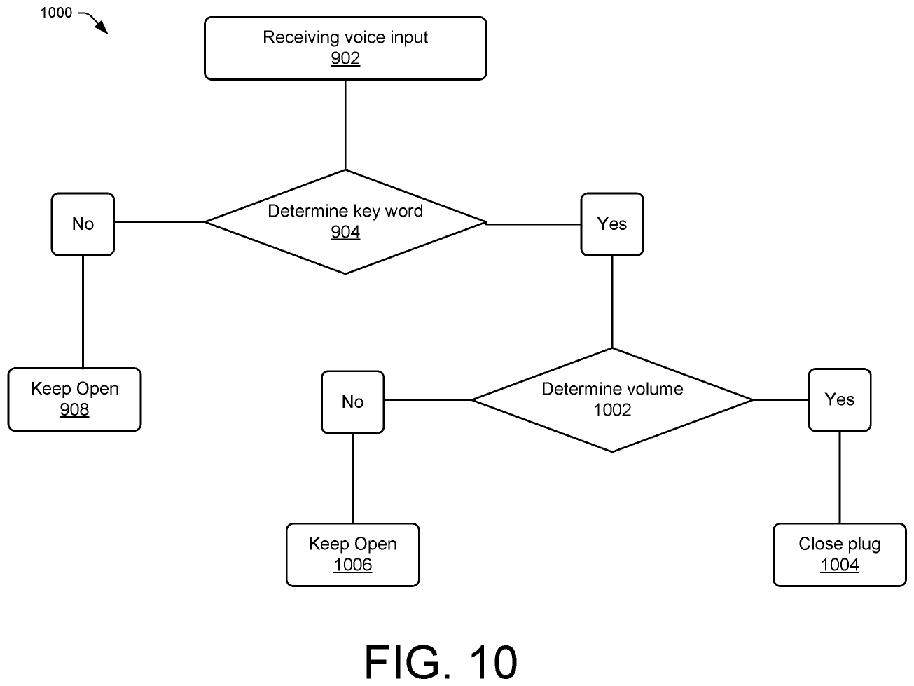

FIG. 10 illustrates another example method 1000 of displacing the earplug between an open position and a closed position according to an embodiment of the instant disclosure. Method 1000 may include operation 902, operation 904, and operation 908. Here, after operation 904, method 1000 may continue with operation 1002. For example, if at operation 904 the microcontroller determines the predetermined keyword is present in the voice input from the hunter, then method 1000 may continue with operation 1002. Operation 1002 represents, determining, by the microcontroller, a threshold volume of the predetermined keyword. In one example, a threshold volume as used herein may comprise about 80 decibels. In another example, a threshold volume as used herein may comprise about 90 decibels. Method 1000 may proceed to operation 1004, if the threshold volume of the predetermined keyword is approaching or at least equal to the threshold volume. For example, if the hunters 102(1) and 102(N) voice the predetermined keyword 112 loud enough to comprise about 80 decibels with regard to the game 104, then method 1000 may proceed to operation 1004. Operation 1004, represents the microcontroller causing the servomechanism to displace the earplug from the open position to the closed position. Alternatively, method 1000 may proceed to operation 1006, if the threshold volume of the predetermined keyword is not approaching or is at least less than the threshold volume. For example, if the hunters 102(1) and 102(N) do not voice the predetermined keyword 112 loud enough to comprise about 80 decibels with regard to the game 104 then method 1000 may proceed to operation 1006. For example, the hunters 102(1) and 102(N) may not voice the predetermined keyword 112 loud enough because the hunters 102(1) and 102(N) may be uttering the predetermined keyword 112 in a normal conversation comprising about 60 decibels. In another example, the hunters 102(1) and 102(N) may not voice the predetermined keyword 112 loud enough because the hunters 102(1) and 102(N) may be uttering the predetermined keyword 112 in a whispered conversation comprising about 30 decibels. Operation 1006 represents the microcontroller causing the servomechanism to refrain from displacing the earplug from the open position to the closed position to remain in the open position.

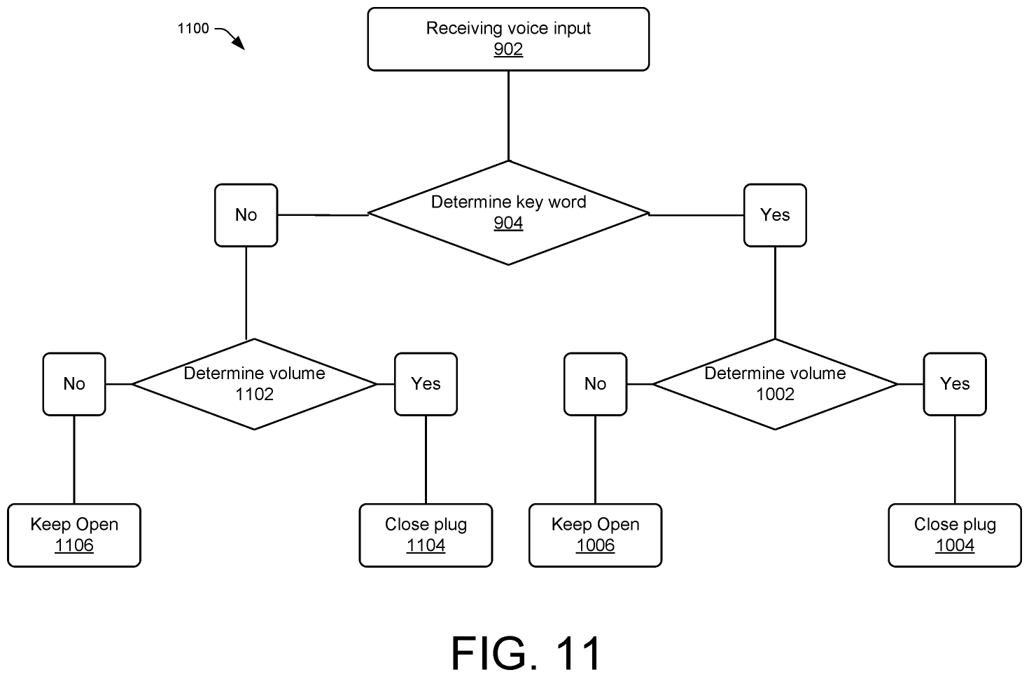

FIG. 11 illustrates another example method 1100 of displacing the earplug between an open position and a closed position according to an embodiment of the instant disclosure. Method 1100 may include operation 902, operation 904, operation 1002, operation 1004, and operation 1006. Here, after operation 904, method 1100 may continue with operation 1102. For example, if at operation 904 the microcontroller determines the predetermined keyword is not present in the voice input from the hunter, then method 1100 may continue with operation 1102. Here, at operation 904, the hunter may have mumbled the predetermined keyword or indistinctly shouted the predetermined keyword causing the microcontroller to determine the predetermined keyword is not present in the voice input from the hunter resulting in method 1100 to continue with operation 1102. Operation 1102 represents, determining, by the microcontroller, a threshold volume of the voiced input (e.g., shouted word) from the hunter. Method 1100 may proceed to operation 1104 if the threshold volume of the voiced input is approaching or at least equal to the threshold volume. For example, if the hunters 102(1) and 102(N) mumbled or indistinctly shouted the predetermined keyword loud enough to comprise about 80 decibels with regard to the game 104, then method 1100 may proceed to operation 1104. Operation 1104, represents the microcontroller causing the servomechanism to displace the earplug from the open position to the closed position. Alternatively, method 1100 may proceed to operation 1106, if the threshold volume of the voiced input is not approaching or is at least less than the threshold volume. For example, if the hunters 102(1) and 102(N) did not shout the mumbled or indistinctly pronounced predetermined keyword loud enough to comprise about 80 decibels with regard to the game 104 then method 1100 may proceed to operation 1106. Operation 1106 represents the microcontroller causing the servomechanism to refrain from displacing the earplug from the open position to the closed position to remain in the open position.

### CONCLUSION

Although the invention has been described in language specific to structural features and/or methodological acts, it is to be understood that the invention is not necessarily limited to the specific features or acts described. Rather, the specific features and acts are disclosed as illustrative forms of implementing the invention. For example, while embodiments are described having certain shapes, sizes, and configurations, these shapes, sizes, and configurations are merely illustrative.

What is claimed is:

1. A hearing protection system comprising:
a receptacle receivable by an ear of a user, the receptacle including a cavity arranged to fit in at least a portion of the ear;
an earplug moveably displaceable between an open position and a closed position,
wherein:
when in the open position, the earplug is positioned adjacent to the cavity of the receptacle to allow the user to hear ambient sound at an unchanged volume, and
when in the closed position, the earplug is positioned over the cavity of the receptacle to at least partly block the ambient sound and allow the user to hear the ambient sound at a reduced volume that is less than the unchanged volume; and
a trigger mechanism, responsive to a voice input, to cause the earplug to displace between the open position and the closed position based at least in part on receiving a predetermined keyword.

2. The hearing protection system of claim 1, wherein the trigger mechanism is further responsive to the voice input, to cause the earplug to displace between the open position and the closed position based at least in part on receiving a threshold volume of the predetermined keyword.

3. The hearing protection system of claim 2, wherein the trigger mechanism causes the earplug to displace from the open position to the closed position in response to receiving the predetermined keyword or receiving the threshold volume of the keyword.

4. The hearing protection system of claim 2, wherein the trigger mechanism causes the earplug to displace from the open position to the closed position in response to receiving the predetermined keyword and receiving the threshold volume of the predetermined keyword.

5. The hearing protection system of claim 1, wherein the voice input is a first voice input, the predetermined keyword is a first predetermined keyword, the trigger mechanism causes the earplug to displace from the open position to the closed position in response to receiving the first predetermined keyword, and
wherein the trigger mechanism is further responsive to a second voice input, to cause the earplug to displace from the closed position to the open position based at least in part on receiving a second predetermined keyword.

6. The hearing protection system of claim 1, wherein the trigger mechanism causes the earplug to remain in the closed position based at least in part on a predetermined amount of time.

7. The hearing protection system of claim 1, wherein the earplug is moveably disposed radially in the plane covering the cavity of the receptacle between the open position and the closed position.

8. The hearing protection system of claim 1, wherein the earplug is moveably disposed linearly in the plane covering the cavity of the receptacle between the open position and the closed position.

9. The hearing protection system of claim 1, further comprising a servomechanism coupled to the earplug, wherein the servomechanism displaces the earplug between the open position and the closed position based at least in part on a wired or wireless signal received from the trigger mechanism.

10. A hearing protection system comprising:
an earplug attachable proximate to an ear of a user; and
a trigger mechanism, responsive to a voice input, to cause the earplug to displace between an open position and a closed position based at least in part on receiving a predetermined keyword, wherein
when in the open position, the earplug is positioned away from the ear of the user to allow the user to hear ambient sound at an unchanged volume, and
when in the closed position, the earplug is positioned proximal to the ear of the user to at least partly block the ambient sound and allow the user to hear the ambient sound at a reduced volume that is less than the unchanged volume.

11. The hearing protection system of claim 10, wherein the trigger mechanism is further responsive to the voice input, to cause the earplug to displace between the open position and the closed position based at least in part on receiving a threshold volume of the predetermined keyword.

12. The hearing protection system of claim 11, wherein the trigger mechanism causes the earplug to displace from the open position to the closed position in response to receiving the predetermined keyword or receiving the threshold volume of the keyword.

13. The hearing protection system of claim 11, wherein the trigger mechanism causes the earplug to displace from the open position to the closed position in response to receiving the predetermined keyword and receiving the threshold volume of the predetermined keyword.

14. The hearing protection system of claim 10, wherein the voice input is a first voice input, the predetermined keyword is a first predetermined keyword, the trigger mechanism causes the earplug to displace from the open position to the closed position in response to receiving the first predetermined keyword, and
wherein the trigger mechanism is further responsive to a second voice input, to cause the earplug to displace from the closed position to the open position based at least in part on receiving a second predetermined keyword.

15. The hearing protection system of claim 10, wherein the trigger mechanism causes the earplug to remain in the closed position based at least in part on a predetermined amount of time.

16. A hearing protection system comprising:
an earplug displaceable from an open position to a closed position in response to a signal received from a trigger mechanism,
wherein the trigger mechanism is responsive to a voice input, to cause the earplug to displace between the open position and the closed position based at least in part on receiving a predetermined keyword.

17. The hearing protection system of claim 16, wherein the trigger mechanism is further responsive to the voice input, to cause the earplug to displace between the open position and the closed position based at least in part on receiving a threshold volume of the predetermined keyword.

18. The hearing protection system of claim 17, wherein the trigger mechanism causes the earplug to displace from the open position to the closed position in response to receiving the predetermined keyword or receiving the threshold volume of the keyword.

19. The hearing protection system of claim 17, wherein the trigger mechanism causes the earplug to displace from the open position to the closed position in response to receiving the predetermined keyword and receiving the threshold volume of the predetermined keyword.

20. The hearing protection system of claim 16, wherein the voice input is a first voice input, the predetermined keyword is a first predetermined keyword, the trigger mechanism causes the earplug to displace from the open position to the closed position in response to receiving the first predetermined keyword, and
wherein the trigger mechanism is further responsive to a second voice input, to cause the earplug to displace from the closed position to the open position based at least in part on receiving a second predetermined keyword.
